(19) United States
(12) Patent Application Publication
(10) Pub. No.: US 2022/0273857 A1
(43) Pub. Date: Sep. 1, 2022

(54) NOVEL TYPE SUPERIOR VENA CAVA CANNULA FOR USE IN EXTRATHORACIC CANNULATION IN OPEN HEART SURGERY

(71) Applicant: ISTANBUL MEDIPOL UNIVERSITESI, Istanbul (TR)

(72) Inventor: Yahya YILDIZ, Istanbul (TR)

(21) Appl. No.: 17/637,219

(22) PCT Filed: Aug. 21, 2020
(86) PCT No.: PCT/TR2020/050740

§ 371 Date: Feb. 22, 2022

(30) Foreign Application Priority Data
Aug. 23, 2019 (TR) 2019/12744

(51) Int. Cl. A61M 1/36; A61M 39/12; A61M 25/00; A61M 25/01

(52) U.S. Cl. CPC A61M 1/3659; A61M 39/12; A61M 25/007; A61M 25/0108; A61M 1/3666; A61M 1/1698

(57) ABSTRACT

Disclosed is a cannula to be used in open heart surgery to connect the patient to the heart-lung machine reliably and quickly by extra thoracic cannulation.

### TECHNICAL FIELD

This invention refers to a cannula to be used in open heart surgery to connect the patient to the heart-lung machine reliably and quickly by means of extra thoracic cannulation.

### PRIOR ART

In traditional practice, the thorax is opened for arterial and venous cannulation in order to perform cardiopulmonary bypass in open heart surgery. In recent years, connection to the heart-lung machine via extra thoracic cannulation is yet another approach commonly adopted in this type of surgery for various reasons.

Extra thoracic cannulation is especially preferred in cases with ailing veins such as a) reoperative cases, b) minimally invasive (small scars) surgery and c) porcelain, aneurysmatic or dissected aorta.

In addition, the widespread use of other technologies (for example, ultrasonography), the success and relative ease of peripheral cannulation contributed to the development of cardiopulmonary support and extra thoracic extracorporeal membrane oxygenation (ECMO). Extra thoracic cannulation methods are frequently in for cardiopulmonary bypass support including emergency circulatory support for patients with cardiogenic shock and respiratory support for patients with seriously impaired gas exchange (lung failure).

In the prior art, it is known that the patients requiring 2 or more open heart surgeries carry a higher risk of morbidity and mortality in comparison to patients undergoing open heart surgery for the first time. In such cases, by means of extra thoracic cannulation, the blood from superior vena cava is transferred to the heart-lung machine via a cannula placed in the superior vena cava without having to open the sternum and, similarly, the venous blood of the patient is transferred to the heart-lung machine via a cannula inserted into the inferior vena cava through the femoral vein. The oxygenated blood is delivered to the patient again by means of the heart-lung pump inside the machine. This way, the deoxygenated blood is removed from the patient, is oxygenated and delivered to the patient again to provide blood circulation. Said procedure can be carried out by placing the cannula into the relevant vein (i.e superior vena cava). Although there are commercially available cannulas for this procedure, the cannula comprised in the prior art is a femoral artery cannula which leads to certain complications when used in the superior vena cava. Especially that the femoral artery cannula is long makes it possible for this cannula to reach inside the superior vena cava when inserted in the left internal jugular vein and after these cannulas are placed, some of the holes on the cannula may remain exposed and cause air leak into the cannula. In order to fix the holes on the cannula to prevent air leak may also lead to other complications and the cannula needs to be pushed forwards or pulled backwards or placed higher in order to prevent the leak or to properly drain the venous blood. Due to all of the above inconveniences, the use of the cannula inside the superior vena cava remains as known in the prior art remains insufficient.

Additionally, the apertures located on the cannulas used in the prior art are placed on the cannulas at random, irregular intervals. Some of these apertures remain inside the vessel wall and apply suction force to the vessel wall since the cannula works at negative pressure. This causes the apertures on the cannula to be blocked by the vessel wall and, since the venous blood is not properly drained, the apertures lose their function.

When all the above reasons are taken into account, there is need for a cannula that can be used in the superior vena cava during extra thoracic cannulation in open heart surgery.

### BRIEF DESCRIPTION OF THE INVENTION

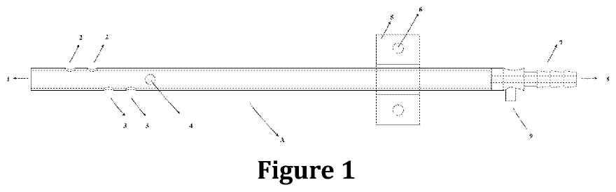

The invention refers to a superior vena cava cannula (A) suitable for use in order to transfer the entire venous blood of the patient to the heart-lung machine by means of extra thoracic cannulation in operations requiring disruption of blood flow in the body, namely in open heart surgery (FIG. 1).

The superior vena cava cannula that is the subject of the invention comprises the proximal end (1), the innominate vein aperture (2), the right subclavian vein aperture (3), the internal jugular vein aperture (4), the adjustable clamp (5), the clamp fixation point (6), air outlet port (7) cardiopulmonary bypass venous connection compartment (8) and distal end (9) compartments.

The superior vena cava cannula with the above details provides efficient blood flow by means of the apertures placed in accordance with the anatomy of the area where they are placed and ensures that the venous blood of the patient is removed from the body without causing damage to the surrounding tissue during cannulation.

### BRIEF DESCRIPTION OF THE FIGURE

A representative drawing of the superior vena cava cannula (A) that is the subject of the invention is provided in FIG. 1.

Reference numbers in the figures and the corresponding parts are listed below;

1: Proximal End

2: Innominate Vein Aperture

3: Right Subclavian Vein Aperture

4: Internal Jugular Vein Aperture

5: Clamp

6: Clamp Fixation Point

7: Air Outlet Port

8: Cardiopulmonary Bypass Machine Venous Connection Compartment

9: Distal End

### DETAILED DESCRIPTION OF THE INVENTION

The superior vena cava cannula that is the subject of the invention is inserted into the right internal jugular vein. Blood flow to the cannula is ensured by apertures located on both sides of the cannula (A); one via the innominate vein by the innominate vain apertures (2); and the other via the right subclavian vein by the right subclavian vein apertures (3). Both apertures also provide blood flow to the cannula through the right internal jugular vein. Blood flow to the cannula through the internal and external jugular veins is ensured by means of the internal jugular vein aperture (4) located on the front and back of the top part of the cannula (A). The adjustable clamp (5) located on the cannula (A) enables the catheter to be fixed on the skin at a desired spot by means of the clamp fixation points (6) located on it. Cardiopulmonary bypass machine venous connection compartment (8) allows the blood flow delivered through the cannula (A) to pass through the distal end (9) and reach the heart-lung machine. The air outlet port (7) is used to discharge the air inside the cannula (A) after it is inserted into the internal jugular vein.

The cannula (A) that is the subject of the invention is single-lumen.

In a preferred embodiment of the invention, the innominate vein apertures (2) located on the sides of the cannula (A) are placed in a single row or in rows of two. In an especially preferred embodiment of the invention, the innominate vein apertures (2) comprise 2-4 apertures placed in two parallel rows.

In a preferred embodiment of the invention, the right subclavian vein apertures (3) located on the sides of the cannula (A) are placed in a single row and they comprise 2 to 4 apertures, i.e 2, 3 or 4 apertures.

The innominate vein apertures (2) and the right subclavian vein apertures on the cannula (A) that is the subject of the invention are separately (3) placed on both sides of the cannula (FIG. 1).

The apertures located on the cannula (A) that is the subject of this invention can be circular or of any other shape.

In an embodiment of the invention, the subclavian vein apertures (3) and the innominate vein apertures (2) located on the cannula (A) can be aligned on both sides of the cannula or with a distance of 2 to 40 mm between them. In a preferred embodiment of the invention, the subclavian vein apertures (3) and the innominate vein apertures (2) were aligned on both sides of the cannula with a distance of 2 to 40 mm between them. In a preferred embodiment of the invention, the (first) innominate vein aperture that is closest to the proximal end located on the cannula, is located at a distance of 0.5 to 5 cm, preferably 0.5 to 2 cm and especially preferably 1 cm from the proximal end (1). In the cannula (A) that is the subject of the invention, the innominate vein apertures (2) are closer to the proximal end (1) than the subclavian vein apertures (3) and, since this location is more suitable for the anatomy of the upper extremity, it enables the cannula to be used more effectively.

The subclavian vein apertures (3) located on the cannula (A) that is the subject of the invention has diameters of 0.5 to 5.0 mm, preferably 0.75 to 3.0 mm, especially preferably 1.0 to 2.5 mm.

The innominate vein apertures (2) located on the cannula (A) that is the subject of the invention has diameters of 0.5 to 5.0 mm, preferably 0.75 to 3.0 mm, especially preferably 1.0 to 2.5 mm.

Said subclavian vein apertures (3) and innominate vein apertures (2) that are the subject of the invention may have different diameters independent of each other within the range of 0.5 to 5.0 mm. In other words, each one of the 2 to 4 subclavian vein apertures specified to be located on the cannula (A) that is the subject of the invention may have different widths within the range of 0.5 to 5.0 mm. The same applies to the innominate vein apertures (2).

In a preferred embodiment of the invention, the internal jugular vein apertures (4) located on the cannula (A) comprise 2 apertures aligned on the front and back of the cannula. The internal jugular vein apertures (4) that are the subject of this invention may have diameters of 0.5 to 3.0 mm or more than 3.0 mm.

The internal jugular vein apertures (4) on the cannula (A) that is the subject of the invention are at a distance of 3.0 to 8.0 cm, preferably 4.0 to 7.0 cm, especially preferably 6 cm from the proximal end (1).

Cardiopulmonary bypass machine venous connection compartment (8) located on the cannula

(A) that is the subject of the invention is 1.5 to 2.5 cm, preferably 2 cm long, and the distal end (9) is located on its end.

The length of the cannula (A) that is the subject of the invention between the proximal end (1) and the distal end (9) are in the range of 13 to 17 cm for an adult patient; for instance, 13 cm, 14 cm, 15 cm, 16 cm or 17 cm.

For pediatric patients, the length of the cannula as well as of the part that will remain inside the internal jugular vein varies by the patient's attributes (height and weight). The length of the cannulas can be in the range of 4 to 8.5 cm for children.

In this respect, the length of the cannula (A) that is the subject of the invention between the proximal end (1) and the distal end (9) can be in the range of 4 to 17 cm. In the prior art, the specialist may use a cannula of a length that is suitable for the physical attributes (height, weight etc.), age and the general condition of the patient. The positioning of the apertures on the cannula as well as the distances between them varies depending on the length of the cannula.

The fact that the apertures located on the frame of the cannula that is the subject of the invention are placed at such intervals as suitable for the anatomy of the upper extremity prevents these apertures from being blocked by the surrounding tissues and ensures proper blood flow.

The superior vena cava cannula (A) that is the subject of the invention may be made of any material compatible with the physiological environment and known to be usable in surgical conditions. In a preferred embodiment of the invention, the superior vena cava cannula (A) that is the subject of the invention may be made of a material selected from among a group comprising polyurethane (PU), polyvinyl chloride (PVC), and silicone, metal wire reinforced silicone.

The superior vena cava cannula (A) that is the subject of the invention may wholly or in part be made of radiopaque material or the said cannula may comprise a radiopaque stripe stretching along the cannula between the proximal end (1) and the distal end (9).

In another aspect, the invention refers to a cannulation kit characterized in that it comprises;
a cannula (A) that comprises the proximal end (1), the innominate vein aperture (2) comprising 2 to 4 holes located on one side of the cannula and placed in a single row or in rows of two, the right subclavian vein aperture (3) comprising 2 to 4 holes located on the other side of the cannula and placed in a single row, the internal jugular vein aperture (4) comprising 2 apertures aligned on the front and the back of the cannula, the adjustable clamp (5), the clamp fixation point (6), air outlet port (7) cardiopulmonary bypass venous connection compartment (8) and distal end (9) compartments.
a dilator that can pass through the cannula (A),
a guidewire,
at least one lancet,
At least one injector.

A cannula (A) to be used in open heart surgery for the extra thoracic cannulation of the patient to the heart-lung machine, comprising the proximal end (1), the innominate vein aperture (2) comprising 2 to 4 holes located on one side of the cannula and placed in a 15 single row or in rows of two, the right subclavian vein aperture (3) comprising 2 to 4 holes located on the other side of the cannula and placed in a single row, the internal jugular vein aperture (4) comprising 2 to 4 apertures aligned on the front and the back of the cannula, the adjustable clamp (5), the clamp fixation point (6), air outlet port (7) cardiopulmonary bypass venous connection compartment (8) and distal end (9) compartments.

For the purposes of this description, the term “comprise” is intended to mean “cover”.

Wherever technically possible, the embodiments of the invention can be combined.

The embodiments are described here in a manner to comprise specific features/components. The description also essentially covers the embodiments comprising said features/components or made up thereof.

Patents, applications and similar technical references are included in this document by reference.

The embodiments specifically and expressly described herein may, solely or in conjunction with one or more other applications, constitute the basis of a waiver.

1. A single-lumen cannula suitable for use in open heart surgery for the extra thoracic cannulation of the patient to the heart-lung machine, comprising a proximal end, an innominate vein aperture comprising 2 to 4 holes located on one side of the cannula and placed in a single row or in rows of two, a right subclavian vein aperture comprising 2 to 4 holes located on the other side of the cannula and placed in a single row, an internal jugular vein aperture comprising 2 to 4 apertures aligned on the front and the back of the cannula, an adjustable clamp, a clamp fixation point, an air outlet port cardiopulmonary bypass venous connection compartment and distal end compartments and wherein the innominate vein aperture closest to the proximal end is located at a distance of 0.5 to 5 cm from the proximal end.

2. A cannula according to claim 1, wherein the subclavian vein apertures and the innominate vein apertures are aligned on both sides of the cannula with a distance of 2 to 40 mm between them.

3. A cannula according to claim 1, wherein the innominate vein apertures are closer to the proximal end than the subclavian vein apertures.

4. A cannula according to claim 1, wherein the subclavian vein apertures have diameters of 0.5 to 5.0 mm.

5. A cannula according to claim 1, wherein the innominate vein apertures have diameters of 0.5 to 5.0 mm.

6. A cannula according to claim 1, wherein the internal jugular vein apertures are located at a distance of 3.0 to 8.0 cm from the proximal end.

7. A cannula according to claim 1, wherein the length of the cardiopulmonary bypass machine venous connection compartment is in the range of 1.5 to 2.5 cm and the distal end is located on its end.

8. A cannula according to claim 1, wherein the cannula is made of a material compatible with the physiological environment and known to be usable in surgical conditions.

9. A cannula according to claim 8, wherein the cannula is made of a material selected from a group consisting of polyurethane (PU), polyvinyl chloride (PVC), silicone, and metal wire reinforced silicone.

10. A cannula according to claim 1, wherein the whole or a part of it is made of a radiopaque material.

11. A cannula according to claim 10, comprising a radiopaque stripe stretching along the cannula between the proximal end and the distal end.

12. A cannulation kit to be used in open heart surgery to connect the patient to the heart-lung machine reliably and quickly by means of extra thoracic cannulation, the kit comprising:
cannula that comprises a aproximal end, an innominate vein aperture comprising 2 to 4 holes located on one side of the cannula and placed in a single row or in rows of two, a right subclavian vein aperture comprising 2 to 4 holes located on the other side of the cannula and placed in a single row, an internal jugular vein aperture comprising 2 apertures aligned on the front and the back of the cannula, an adjustable clamp, a clamp fixation point, an air outlet port cardiopulmonary bypass venous connection compartment and distal end compartments and wherein the innominate vein aperture closest to the proximal end is located at a distance of 0.5 to 5 cm from the proximal end,
a dilator that can pass through the cannula,
a guidewire,
at least one lancet, and
at least one injector.

13. A cannula for use in open heart surgery for the extra thoracic cannulation of the patient to the heart-lung machine, comprising a proximal end, an innominate vein aperture comprising 2 to 4 holes located on one side of the cannula and placed in a single row or in rows of two, a right subclavian vein aperture comprising 2 to 4 holes located on the other side of the cannula and placed in a single row, an internal jugular vein aperture comprising 2 apertures aligned on the front and the back of the cannula, an adjustable clamp, a clamp fixation point, an air outlet port cardiopulmonary bypass venous connection compartment and distal end compartments and wherein the innominate vein aperture closest to the proximal end is located at a distance of 0.5 to 5 cm from the proximal end.

14. (canceled)

15. (canceled)
